(19) United States
(12) Patent Application Publication
(10) Pub. No.: US 2015/0000652 A1
(43) Pub. Date: Jan. 1, 2015

(54) GAS DELIVERY UNIT AND BREATHING MASK FOR DELIVERING RESPIRATORY GAS OF A SUBJECT

(71) Applicant: General Electric Company, Schenectady, NY (US)

(72) Inventor: Heikki Haveri, Huhmari (FI)

(73) Assignee: General Electric Company, Schenectady, NY (US)

(21) Appl. No.: 13/927,500

(22) Filed: Jun. 26, 2013

(51) Int. Cl. A61M 16/08; A61M 16/00; A61M 16/06; A61M 16/14

(52) U.S. Cl. CPC A61M 16/0891; A61M 16/0833; A61M 16/14; A61M 16/0057; A61M 16/06
USPC 128/202.27

(57) ABSTRACT

A gas delivery unit for delivering a respiratory gas of a subject is disclosed herein. The gas delivery unit includes an expiratory limb for an expiratory gas, and an inspiratory limb for inspiratory gas. The gas delivery unit also includes a common limb connecting at a branching point with the expiratory and inspiratory limb for delivering the expiratory and inspiratory gas, and at least one port for a fluid dispenser. The port opens into at least one of the inspiratory limb, the expiratory limb, the common limb and the branching point. The port is along an opening direction forming an angle δ, which is less than 90° degrees, with one of the inspiratory limb, the expiratory limb and the common limb, and which inspiratory limb can form an angle β, which is at an angle of 100°-180° degrees, with the common limb.

### BACKGROUND OF THE INVENTION

This disclosure relates generally to a gas delivery unit and a breathing mask for delivering respiratory gas of a subject.

Tidal volume (TV) is an amount of an air inspired or taken into the lungs in a single breath. TV is also dependent on the sex, size, height, age and a health etc. of a patient. In general TV also decreases as the size of the patient decreases. In an average healthy adult, TV is about 400-600 ml whereas in an average healthy neonate, that measures 3.5-4 kg and is 50 cm tall. TV is approximately 25-50 ml. On the other hand, in an average premature neonate that measures only 500 grams TV is only about 2-3.5 ml. TV of a smaller patient's is very difficult to measure, but it can be approximated to 4-7 ml/kg, applying a general rule of thumb for approximating the TV of the human lung. In practice the TV of the patient suffering pulmonary system deficiency is normally less than the approximation gives.

Patients can be mechanically ventilated invasively or non-invasively. In invasive ventilation an endotracheal tube is placed into a trachea so that it goes through oral or nasal cavity and larynx. In tracheostomy endotracheal tube goes straight into trachea through neck. The other end of the endotracheal tube is connected to a breathing circuit Y-piece through a luer type connector.

Continuous Positive Airway Pressure (CPAP) is a one type of non-invasive positive pressure ventilation used to maintain an elevated baseline respiratory system pressure during spontaneous breathing. Neonates or infants are preferential nose breathers until 5 months of age, which easily facilitates the application of nasal CPAP for a variety of clinical conditions including respiratory distress syndrome, apnea of prematurity and in other conditions that require positive pressure. This is accomplished by inserting nasopharyngeal tubes, affixing nasal prongs, or fitting a nasal mask to the patient.

Common for all, ventilation Methods is that during an inspiration the flesh breathing gas including higher oxygen (O₂) concentration should flow into the patient's lungs through a breathing circuit, nasopharyngeal tubes, affixing nasal prongs, or a nasal mask, then to oral or nasal cavities, a trachea, a bronchus, a bronchi, bronchioles and finally reaching an alveoli deep in the lungs, where all the gas exchange actually occurs. Carbon dioxide (CO₂) molecules in hemoglobin of a blood flowing in tiny blood vessels around the alveoli are replaced with O₂ molecules in the fresh breathing gas through the thin walls of the alveoli. O₂ molecules take their place in the hemoglobin, Whereas CO₂ molecules flow out from the patient within the used expired breathing gas, through the same path as the fresh gas came in during the inspiration. This path common for inspiratory and expiratory gases inside the patient's respiratory system causes rebreathing of gases and is called anatomical dead volume.

The anatomical dead volume is almost impossible to reduce, but it is proportional to the size and the physical condition of the patient. The mechanical dead volume outside the patient depends on a breathing circuit design, an inner diameter of a tubing, connectors and additional accessories, such as sidestream and mainstream gas analyzers connected to patient's respiratory system. Obviously the mechanical dead volume is more critical for smaller patients with smaller TV or patients suffering barotraumas etc., which also decrease TV.

A nebulizer is a device used to administer medicine in the form of a mist of small droplets into the lungs of through the lungs into the blood stream. Nebulizers are commonly used for the treatment of cystic fibrosis, asthma, COPD and other respiratory diseases. Nebulizers use oxygen, compressed air or ultrasonic power to break up medical solutions and suspensions into small droplets that can he delivered from the device into the patient's lungs. With mechanically ventilated patients nebulizers are commonly connected between the inspiratory tubing to increase the delivery efficiency of medication into the lungs, since conventional nebulizers generate mist of droplets continuously, also during the expiratory phase, which is lost into the breathing circuit.

FIG. 1 shows a schematic View of a commonly used setup how nebulizer is connected to a mechanically ventilated patient. The intubated patient 200, has an endotracheal tube 201 placed into the trachea, which other end connects to a endotracheal tube connector 202, which connects to a breathing, circuit L-piece 203, which further connects to a common limb 204 of a breathing circuit Y-piece 205. If additional care devices, such as gas analyzers, are used they are usually placed between the L-piece 203 and Y-piece 205. The Y-piece 205 also comprises inspiratory limb 206 connected to inspiratory tubing 210 and expiratory limb 207 connected to expiratory tubing 211 of a ventilator 220. The common limb 204, inspiratory limb 206 and expiratory limb 207 connect to each other through a connection point 208 allowing inspiratory air to flow from the ventilator 220, through the inspiratory tubing 210, inspiratory limb 206, common limb 204, L-piece 203, endotracheal tube 202 and endotracheal tube 201 into the lungs of the patient 200. The expiratory air flows out from the patient's lungs the same path, but through the expiratory limb 207 and expiratory tubing 211 to the ventilator 220. L-pieces are commonly used for example for usability reasons to direct the inspiratory and expiratory tubing towards the ventilator to prevent them to twist the patient or the endotracheal tube, which may bent and obstruct the endotracheal tube or even disconnect it from the patient. It is also possible to use L-piece for open or closed suctioning when it comprises a port for suctioning tube to enter the breathing circuit. Nebulizers 230 are commonly connected between the inspiratory tubing 210 through a T-piece 231, which allows the mist of droplets, generated by the nebulizer 230, to penetrate the inspiratory air through a nebulizer limb 232 of a T-piece 231.

The mist form medicine is formed of small fluid drops having a diameter conventionally between 0.1-100 μm depending on the nebulizing technology used. The fluid drops are commonly sprayed out from the nebulizer with a speed, which is specific to used nebulizing technology. The smallest drops have lower inertia proportional to the lower mass and the speed of droplets, which slows down faster due air resistance. The largest drops have higher inertia, proportional to the higher mass and the higher speed of droplets, which also maintain their velocity longer although the air resistance slows down the speed finally.

It is commonly recommended that the best delivery efficiency may be achieved with droplets having a diameter of 1-5 μm, which most probably penetrate the inspiratory air flow and then float down and reach the alveoli in the deep lung. The optimum sized droplet, between 1-5 μm are difficult to generate, but can be achieved with for example nebulizers based on vibrating mesh plate technology.

When the smaller drops are sprayed towards the inspiratory gas flow through the nebulizer limb 232 in FIG. 1, rather than penetrating into the flow, they tend to bounce back from the gas flowing by after which they tend to continue to float in the volume at the nebulizer output in the limb 232 never reaching the inspiratory flow neither the lungs.

When the largest drops are sprayed towards the inspiratory gas flow they penetrate the gas flow easily and may start to float within the inspiratory gas flow towards the patient. However as the larger drops have higher inertia they also tend to continue to travel into their original direction, thus hitting the walls of a breathing circuit, especially in places where the cross sectional area of the flow path changes rapidly in the connections between conventional breathing circuit parts, such as conventional catheter mounts, L-, T-, Y-pieces, conventional flow, pressure, gas analyzing devices etc. or in the places of very high constrictions, such as where the breathing circuit connects with the endotracheal tube through an endotracheal tube connector. These connections also generate turbulences into the gas flow that redirects the drops floating within the gas flow causing them to hit the breathing circuit wall, but also to collide and combine with each other forming larger drops with higher inertia and incorrect flow directions causing them to hit the walls of the breathing circuit.

The outer diameter Of endotracheal tube is Selected to fit into the patient's trachea to prevent gases to leak through the connection. The inner diameters of endotracheal tubes vary between 2-10 mm in 0.5 mm steps depending on the size of a patient. Every size endotracheal tube connects to the similar sized endotracheal tube port of the endotracheal tube connector. The endotracheal tithe connector further connects to the rest of the breathing circuit through the standard sized breathing circuit port of the endotracheal tube connector. The standard breathing circuit ports of endotracheal tube connectors have outer diameters of 8 mm, 15 mm and 22 mm, but the connector with 15 mm outer diameter is the most commonly used. This means that the cross sectional area between the endotracheal tube end and the breathing circuit end of the endotracheal tube connector changes rapidly and the difference in cross sectional area increases when the patient s size decreases. When the breathing circuit with an inner diameter of 15 mm is connected to for example endotracheal tube with the inner diameter of 2 mm through the endotracheal tube connector it generates a huge cross sectional change into the flow path. This increases the gas flow speed and thus the inertia of the drops floating within the gas considerably, but also the direction of the gas flow near the walls of the connector. The rapid, conical change also generates strong flow turbulences scattering the direction of drops, which in turn causes the drops to collide and combine with each other and to hit walls of an endotracheal tube connector.

In the conventional system shown in FIG. 1 the larger drops will hit the breathing circuit walls, first the wall of the T-piece 231 opposite to the opening of the limb 232, then the wall of the Y-piece 205 opposite to the opening of the inspiratory limb 206, then the wall of the L-piece 203 in the sharp turn and then the walls of the endotracheal tube connector 202 where the cross sectional area changes rapidly when it connects into the endotracheal tube with smaller diameter, thus newer really reaching the alveoli deep in the lungs.

It is not reasonable to place the nebulizer 230 between the expiratory limb 207 or the expiratory tube 211, since the mist of drops generated would be lost into the expiration gas flowing away from the patient.

The nebulizer 230 may be place between the endotracheal tube connector 202 and the common limb 204, which increases the delivery efficiency of even a continuously functioning nebulizer compared to the placement into the expiratory side since the part of the mist of drops produced during inspiration will flow towards the patient's lungs, but this place would increase the volume common for inspiratory and expiratory air flow increasing the rebreathing of gases, which is fatal for the gas exchange in the lungs especially with smaller size patients whose tidal volumes are small.

If the nebulizer 230 is placed between the inspiratory limb 206 or the inspiratory rube 210 the efficiency of delivery increases compared to the placement into the expiratory side since the part of the mist of drops produced during inspiration will flow towards the patient's lungs even the nebulizer produces the drops continuously. However the number of connections, constrictions and turbulences between the nebulizer and the patient lungs increases decreasing the number of drops reaching the lungs. Also the distance between the nebulizer and the patient's lungs becomes longer causing the drops to collide and combine with each other forming larger droplets, which hit the walls of the breathing circuit more probably.

Thus at the moment there does not exist an efficient way of delivering medicine into the patient's lungs as a mist of droplets. For this reason the expensive drugs are lost into the breathing circuit and devices connected to it newer reaching the patient's lungs, Which makes the patient care very problematic as there is no understanding how much drug ends up into the patient lungs and how appropriate and effective the planned care was.

### BRIEF DESCRIPTION OF THE INVENTION

The above-mentioned shortcomings, disadvantages and problems are addressed herein which will be understood by reading and understanding the following specification.

In an embodiment, a gas delivery unit for delivering a respiratory gas of a subject includes an expiratory limb for delivering an expiratory gas, and an inspiratory limb for delivering, an inspiratory gas. The gas delivery unit also includes a common limb connecting at a branching point with the expiratory limb and the inspiratory limb for delivering both the expiratory gas and the inspiratory gas, and at least one port for a fluid dispenser. The port is configured to open into at least one of the inspiratory limb, the expiratory limb, the common limb and the branching point, the port having a longitudinal axis along an opening direction, and which longitudinal axis of the port is configured to form an angle δ, which is less than 90° degrees, with a longitudinal axis of one of the inspiratory limb, the expiratory limb and the common limb, and which longitudinal axis of the inspiratory limb is configured to form an angle β, which is at an angle of 100°-180° degrees, with the longitudinal axis of the common limb.

In another embodiment, a breathing mask for delivering a respiratory gas of a subject includes an expiratory limb for delivering an expiratory gas, and an inspiratory limb for delivering an inspiratory gas. The breathing mask also includes a common limb connecting at a branching point with the expiratory limb and the inspiratory limb for delivering both the expiratory gas and the inspiratory gas, and at least one port for a fluid dispenser. The port is configured to open into at least one of the inspiratory limb, the expiratory limb, the common limb and the branching point, the port having a longitudinal axis along an opening, direction, and which longitudinal axis of the port is configured to form an angle δ, which is less than 90° degrees, with a longitudinal axis of one of the inspiratory limb, the expiratory limb and the common limb, and which longitudinal axis of the inspiratory limb is configured to form an angle β, which is at an angle of 100°-180° degrees, with the longitudinal axis of the common limb.

In yet another embodiment, a breathing mask for delivering a respiratory gas of a subject includes an expiratory limb for delivering an expiratory gas, and an inspiratory limb for delivering an inspiratory gas. The breathing mask also includes a common limb connecting at a branching point with the expiratory limb and the inspiratory limb for delivering both the expiratory gas and the inspiratory gas, and at least one of only one respiratory tube and at least two respiratory tubes, the common limb being in operational contact with one of the only one respiratory tube and the at least two respiratory tubes for delivering inspiratory gas from the common limb into at least one cavity of the subject and expiratory gas from the at least one cavity of the subject to the common limb. The breathing mask also includes at least one port for a fluid dispenser, which port is configured to open into at least one of the inspiratory limb, the expiratory limb, the common limb and the branching point. A longitudinal axis of the port is along an opening direction, and which longitudinal axis of the port is configured to form an angle δ, which is less than 90° degrees, with a longitudinal axis of one of the inspiratory limb, the expiratory limb and the common limb, and which longitudinal axis of the inspiratory limb is configured to forum an angle β, which is at an angle of 100-180° degrees, with the longitudinal axis of the common limb. A diameter of the common limb is configured to deviate less than 10% from one of a diameter of the only one respiratory tube and combined diameters of the at least two respiratory tubes.

Various other features, objects, and advantages of the invention will be made apparent to those skilled in art from the accompanying drawings and detailed description thereof.

### BRIEF DESCRIPTION OF THE DRAWINGS

FIG. 1 shows a perspective schematic view of a prior art breathing circuit with a nebulizer:

FIG. 2 shows a schematic perspective view of a gas delivery unit connectable to a fluid dispenser in accordance with an embodiment;

FIG. 3 shows a perspective, schematic view of the breathing circuit incorporating the gas delivery unit of FIG. 2;

FIG. 4 shows a perspective, schematic view of the breathing mask connectable to a fluid dispenser in accordance with an embodiment; and

FIG. 5 shows a perspective schematic view of the breathing circuit including a breathing mask of FIG. 4 attached to nasal cavities of a subject.

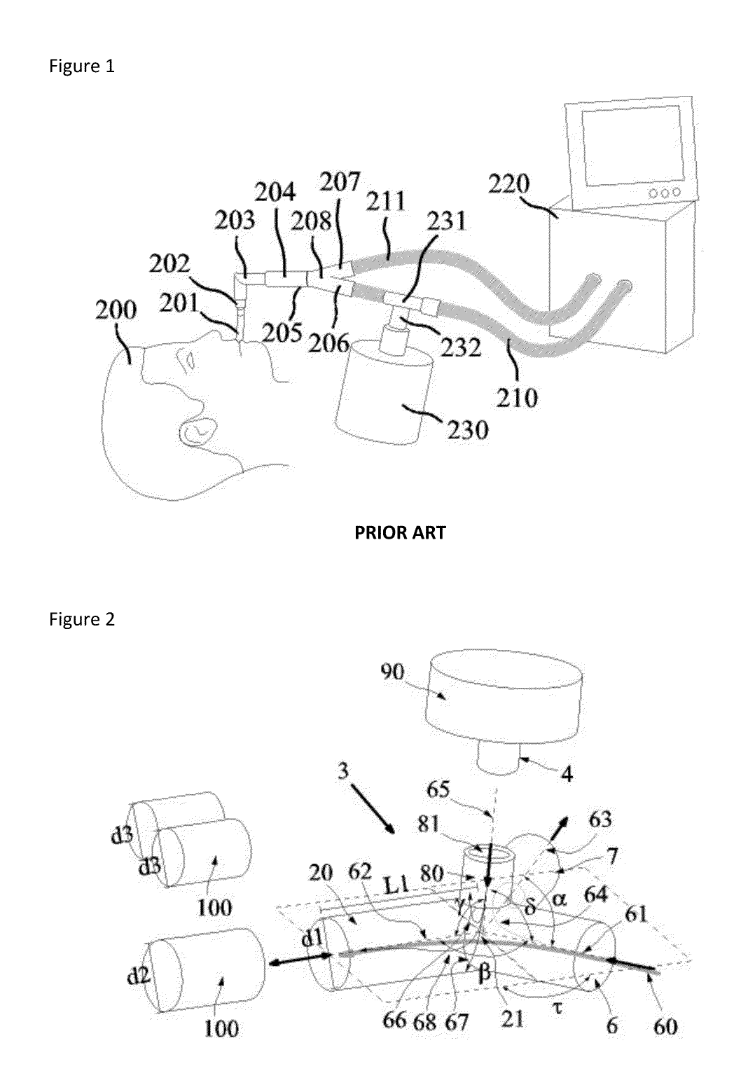

### DETAILED DESCRIPTION OF THE INVENTION

Specific embodiments are explained in the following detailed description making a reference to accompanying drawings. These detailed embodiments can naturally be modified and should not limit the scope of the invention as set forth in the claims.

FIG. 2 shows a simplified schematic view of a gas delivery unit 3, such as a. breathing circuit branching unit 3 used to guide inspiratory and expiratory air during ventilation of a patient and to allow the delivery of fluids into the patient's lungs during inspiration. The gas delivery unit 3 comprises a branching point 21, such as connection point, that connects an inspiratory limb 6 for inspiratory gas, an expiratory limb 7 for expiratory gas, a common limb 20 for both the inspiratory and the expiratory gases and at least one port 81 such as pressure saving port, with or without an additional limb 80, for connecting a fluid dispenser 90, such as a liquid dispenser, to deliver fluids into the patient's lungs during inspiration. The port with or without the additional limb 80 has a longitudinal axis 65 along its opening direction. Thus the port may open through the additional limb 80 haying same longitudinal axis 65 with the port into at least one of the inspiratory limb, the expiratory limb, the common limb and the branching point. Typical fluid dispenser is a nebulizer or a humidifier. The additional limb 80 is not quite necessary meaning that only the port can be practical, too. This is especially in case the fluid dispenser can be provided with very short connection part 4, which does not enter deep into the gas delivery unit disturbing the flow therein. During the inspiration the inspiratory gas 60 flows through the inspiratory limb 6, past the expiratory limb 7 and the at least one port 81 with or without the additional limb 80, through the common limb 20 into the patient's lungs. During the expiration the expiratory gas flows out from the patient's lungs through the common limb 20, past the at least one port 81 with or without the additional limb 80 and the inspiratory limb 6 finally through the expiratory limb 7.

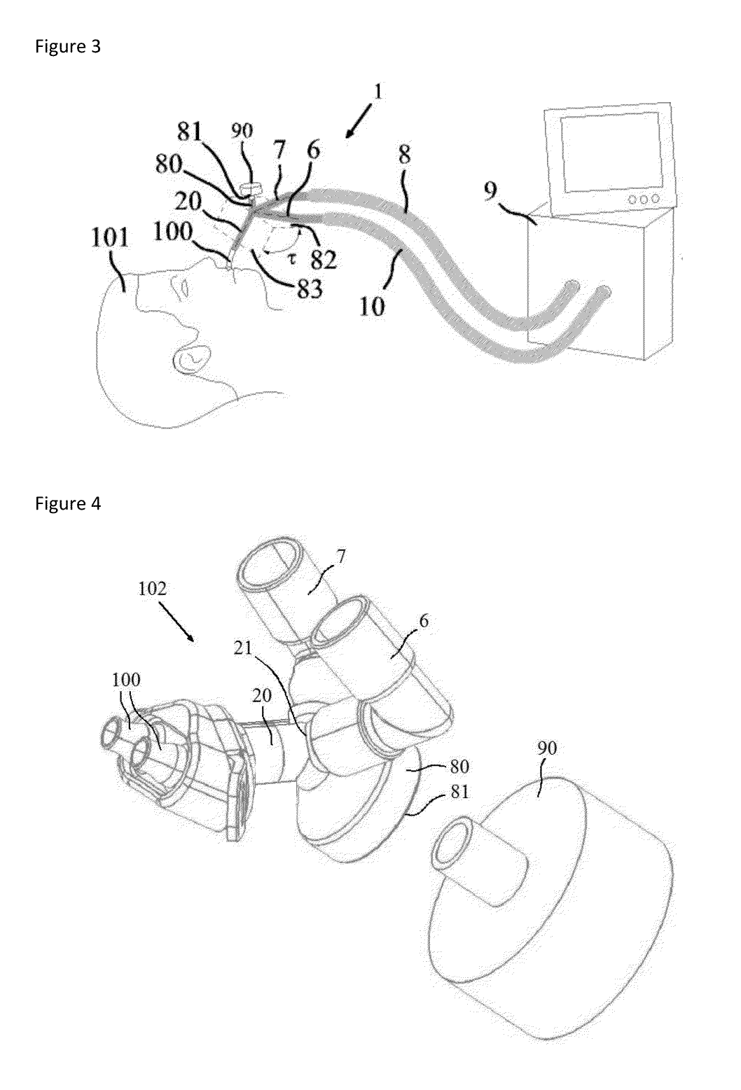

The at least one port 81 enable connecting or disconnecting the fluid dispenser 90 or any other respiratory care device advantageously without losing the pressure in the breathing circuit 1 as shown in FIG. 3 that keeps the lungs and alveoli open for ventilation. The fluid dispenser is used for delivering mist form medicine or water through the at least one port 81 and possibly through the additional limb 80, if such exists, to mix the mist into the inspiratory gas flowing by the port 81. The inspiratory gas flow 60 then carries the mixture of gas and mist form medicine through the common limb 20 into the patient's lungs. It is not desirable to deliver the mist form medicine into the expiratory gas flow flowing out from the patient, thus to increase the delivery efficiency considerably the fluid delivery is advantageously turned on only for the time between inspiration and turned off for the time between expiration to prevent the mist to escape within the expiratory gas flow through the expiratory limb 7 into the expiratory breathing circuit tube $ and the ventilator 9 as shown in FIG. 3.

It is advantageous to place the fluid dispenser as close to the patient's airways as possible to minimize the distance between the fluid dispenser and the lungs, but also to minimize the numerous mechanical connections between different breathing circuit parts, turns, intersections etc. to enable as straightforward and smooth flow path as possible for the drops to travel from the fluid dispenser 90 into the patient's lungs, preventing the drops to collide with each other and hit the walls of a breathing circuit. In the schematic view of the gas delivery unit 3 of the breathing circuit 1 shown in FIG. 2 the at least one port 81 with or without the additional limb 80 connects to the patient's airways through the Common limb 20 and the at least one respiratory tube 100, such as an endotracheal tube or a nasal tube, forming a straight and smooth flow path with a minimal distance and with the minimal number of mechanical connections and minimal changes in the cross sectional area, ensuring that the minimum number of drops hit the breathing circuit walls due their inertia. Preferably the diameter d1 of the common limb 20 is substantially same, typically deviating less than 10% from the diameter d2 of one respiratory tube 100 or the combined diameters d3 of several respiratory tubes 100 in case the number of respiratory tubes is higher than 1, for example 2 for both nasal cavities of the patient. Also the turbulences in the flow path are minimized, which otherwise make the drops to deviate from their original flow path and collide with each other. Also the minimum length of the flow path ensures that the minimum amount of drops collide and combine with each other avoiding them to form larger drops with higher inertia that then would hit the breathing circuit walls.

The standard endotracheal tube connector, between the standard size breathing circuits and the standard endotracheal tubes, gather a lot of drops into its walls decreasing the delivery efficiency of the mist form medicine. The volume of the whole breathing circuit common for inspiratory and expiratory gases is also vast causing rebreathing of gases, which is especially problematic with smaller sized patients. Thus it is advantageous in many ways to have patient size specific breathing circuit gas delivery units 3 with an inner diameter similar to patient size specific endotracheal tubes or nasal tubes 100, which they connect to with a minimal change in a cross sectional area firming a continuous and uniform flow path between the at least one port with or without the additional limb SO and the at least one respiratory tube 100, but also ensuring a minimal volume common for the inspiratory and expiratory gases minimizing the rebreathing of gases.

The best results are achieved when the fluid dispenser is placed outside the common limb 20 to minimize the volume causing rebreathing of gases, but also outside the inspiratory limb 6, as close to the patient as possible, to minimize the distance to the patient's lungs and the number of connections, turns and intersections to minimize the number of collisions between the drops and number of collisions into the walls of the breathing circuit tubing. Thus the branching point 21 between the inspiratory limb 6 and the common limb 20 enables the most efficient delivery of drops into the patient's lungs with a minimal breathing circuit volume causing rebreathing of gases.

The fluid dispenser 90 may be connected through the port 81, located in the inspiratory limb 6 or in the common limb 20 (not shown in figures) to allow nebulized fluid drops to penetrate and flow within the inspiratory gas flow. However if the fluid dispenser is placed in the inspiratory limb 6 the distance and the number of connections between the fluid dispenser and the patient increases. The longer the distance the more probably the fluid drops collide and combine with each other forming larger droplets with higher inertia after they hit the walls of the breathing circuit as in the turn. If the fluid dispenser is placed in the common limb 20 the volume of a common path, where the inspiratory and expiratory gases flow, is increased causing rebreathing of gases. If the nebulizer is placed in the expiratory limb 7 it is more difficult for the mist of drops to enter the inspiratory flow and most of the mist will be lost during expiration. However, it is possible to place the port $1 with or without the additional limb 80 into the expiratory limb 7, but then advantageously the port with or without the additional limb $0 may locate close to the branching point 21, typically within a distance which is less than the diameter of the expiratory limb 7.

The inspiratory gas flow in the breathing circuit needs to be laminar for the fluid drops to travel straightforward on average towards the patient lungs within the inspiratory gas flow. Laminar inspiratory gas flow also enables easier penetration of drops into the gas flow and enables them to retain the longitudinal flow direction in the common limb 20 and endotracheal tube or nasal tubes 100 enabling them to float within the inspiratory gas flow into the patient's lungs without colliding with each other and into the walls of the breathing circuit. Every cross sectional change, turn or intersection generates turbulences along the inspiratory gas flow path. The inspiratory air 60 flowing through the inspiratory limb 6 into the common limb 20, past the expiratory limb 7 generates turbulences 64 into the output of expiratory limb 7 near the branching point 21. These flow turbulences disturb the penetration and the direction of droplets sprayed by the fluid dispenser through the port 81 with or without the additional limb 80 into the inspiratory gas flowing by. The turbulences 64 generated into the output of expiratory limb 7 near the branching point 21 can be minimized by adjusting the angle a between the longitudinal axis 61 of the inspiratory limb 6 and the longitudinal axis 63 of the expiratory limb 7 and the angle β between the longitudinal axis 61 of the inspiratory limb 6 and the longitudinal axis 62 of the common limb 20. Thus it is advantageous that the angle β is between 100°-180° degrees and the angle a is between 0°-170° degrees. The better results are achieved, with the angle β between 110°-160° degrees and the angle α is between 30°-120° degrees and the best results are achieved with the angle β between 125°-145° degrees and the angle a is between 45°-100° degrees. When the angles α and β are 0° degrees they represent a special case of coaxial tubing when the inspiratory tubing, is located inside the expiratory tubing.

When considering usability aspects it is advantageous that the inspiratory limb 6 and expiratory limb 7 open out parallel towards the ventilator 9 and that they are bent into an angle τ in regard to common limb 20 to avoid twisting forces from the inspiratory and expiratory tubes directed to the endotracheal tube to prevent it to disconnect from the patients trachea as shown in FIG. 3. In other words the longitudinal axis 61 of the inspiratory limb 6 and the longitudinal axis 63 of the expiratory limb 7 may form a first plane 82 as shown in FIG. 3 and the longitudinal axis 62 of the common limb 20 may form a second plane 83, which second plane is at an angle τ with the first plane 82. Typically the angle τ is deviating from 180° degrees. It is also advantageous that the at least one port 81 with or without the additional limb 80 is not directed parallel between the inspiratory and expiratory limbs 6 and 7, but rather away from those limbs, since the fluid dispenser 90 needs a certain device space to fit into the port 81 in the additional limb 80 between the inspiratory limb 6 and expiratory limb 7 as shown in FIG. 3. It is advantageous that the angle τ is between 90°-170° degrees, but the better results are achieved with the angle τ between 100°-160° degrees and the best results with the angle τ between 120°-145° degrees.

The inertia of droplets needs to be appropriate to enable the droplets to penetrate into the inspiratory gas flow 60 and to prevent droplets bouncing from the boundary surface of inspiratory gas flow. Fluid dispensers based on vibrating mesh plate can produce droplets between 1-5 μm with a fairly constant droplets speed. Also the average speed of inspiratory gas flow between different sizes of patients can be equalized with the patient size specific breathing circuit gas delivery units 3, shown in FIG. 2. In the schematic drawing in FIG. 2 the contact angle δ between the longitudinal axis 65 the opening direction of the port 81 with or without the additional limb 80 and the longitudinal axis 61 of the inspiratory limb 6 as well as the contact angle γ between the longitudinal axis 65 of the opening direction of the at least one port 81 with or without the additional limb and the longitudinal axis of the common limb 20 can be adjusted to direct the droplets to penetrate into the inspiratory gas flow. If the contact angles δ and γ are too low or too high the droplets with less inertia will bounce back from the inspiratory air 60 as shown with the line 66. When the contact angles δ and γ are close to perpendicular the droplets with higher inertia will penetrate through the inspiratory air flow 60 and hit the wall on the opposite side of the additional limb 80 as shown with the line 67 in FIG. 10. With the correct contact angles δ and γ adjusted to correspond the inertia of droplets having the optimum diameter between 1-5 μm and the muzzle velocity specific to used nebulizer technology the droplets will penetrate the inspiratory air flow 60 and continue into the direction of the flow as shown with the line 68. The contact angles δ and γ depend on the angles α, β and τ. Thus it is advantageous that the angle δ is between 10°-90° degrees and the angle y is between 90°-170° degrees. The better results are achieved with the angle δ between 20°-80° degrees and the angle γ between 100°-160° degrees and the best results are achieved with the angle δ between 30°-70° degrees and the angle γ between 110°-150° degrees.

The length L1 of the common limb 20 shown in FIG. 2 can be adjusted to allow the drops travelling for example the path 68 to redirect with the inspiratory air flow flowing along the longitudinal axis 62 of the common limb 20 before entering the endotracheal tube.

Thus the optimum place for the fluid dispenser 90 to administer medicine into the patient's lungs in the form of mist or droplets is to connect it into the at least one port 81 with or without the additional limb 80, which is placed close to or into the branching point 21 of the gas delivery unit 3 firstly to minimize the distance between the fluid dispenser 90 and the patient, secondly to minimize the number of turns, intersections, constrictions and mechanical connections between the different breathing circuit parts, thirdly the location of the port 81 with or without the additional limb 80 and the direction of the port with or without the additional limb are adjusted in regard to longitudinal axes of one of the inspiratory limb 6, expiratory limb 7 and common limb 20 to enable the penetration of droplets into inspiratory gas flow and not into the walls of the breathing circuit and fourthly the location and the direction of the longitudinal axes of at least one of the additional limb 80 and the at least one port 81 is adjusted in regard to longitudinal axes of inspiratory limb 6 and expiratory limb 7 to increase the usability of the gas delivery unit 3, but also to minimize the flow turbulences make the inspiratory air flow laminar and suitable for the droplets to float into the correct direction and inertia along the longitudinal axes of the common limb 20 into the patient's lungs.

The offset in distance in the intersection between the longitudinal axes 65 along at least one of the opening of the port 81 and along the longitudinal axis of the additional limb 80 and the common limb 20 can be used to generate controlled flow swirl that may ease the penetration of droplets into the inspiratory gas flow and increase the delivery efficiency.

A breathing mask 102 is shown in FIG. 4 comprising mainly same elements and similar principle as the gas delivery unit 3 explained hereinbefore, which makes the breathing mask a special example of the gas delivery unit. This is the reason why same reference numbers are used in FIG. 4 as in FIG. 2 or 3. The breathing mask may comprise at least one respiratory tube 100. For nasal cavities respiratory tubes 100 are needed two, one for each nasal cavity. For oral cavity at least one, but typically only one respiratory tube is necessary. The at least one respiratory tube 100 is in operational contact with the common limb 20. Thus the at least one respiratory tube provides inspiratory gas from the common limb to either the nasal cavities of the subject or to the oral cavity of the subject. Further the at least one respiratory tube provides expiratory gas from either the nasal cavities or the oral cavity or both of them to the common limb.

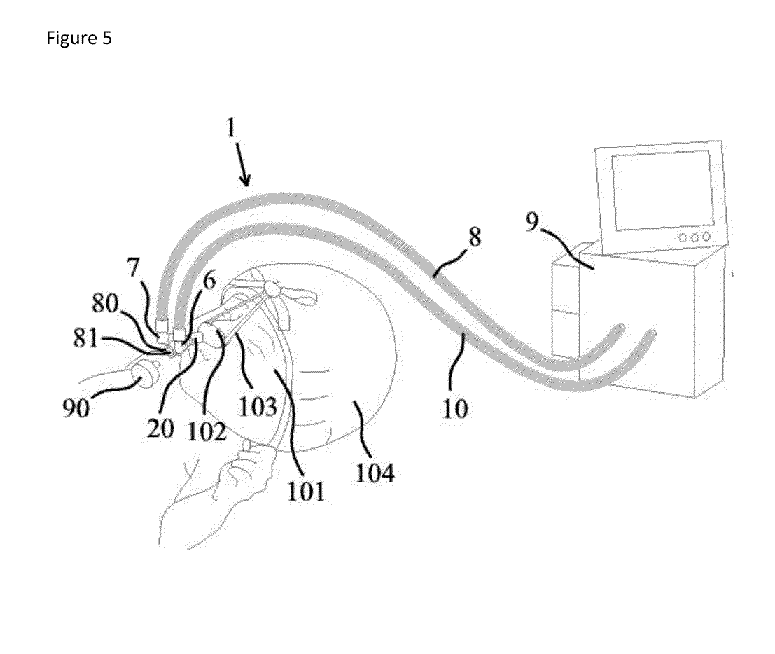

FIG. 5 shows an embodiment of the breathing circuit 1 comprising the breathing mask 102 of FIG. 4 attached to the nasal cavities of the patient 101. Also in this Figure same reference numbers have been used as in FIGS. 2, 3 and 4. The respiratory tubes 100 of the breathing mask guide the inspiratory gas flow from the ventilator 9 along an inspiratory breathing circuit tube 10 to the inspiratory limb 6 and further to the common limb 20 and then to the patient. Correspondingly the expiratory gas flow from the patient is guided through the respiratory tubes 100 to the common limb 20 and further through the expiratory limb 7 and the expiratory breathing circuit tube 8 to the ventilator 9. The fluid dispenser 90 may be attached to the port 81 with or without the additional limb 80 to dose the fluid to the gas flow for the patient breathing. The breathing mask 102 is attached against patient's nostrils and may be kept in place with a. flexible string 103 attached to a cap 104 or similar. Naturally the breathing mask could also cover the mouth, but in this specific embodiment it is not necessarily needed.

The written description uses examples to disclose the invention, including the best mode, and also to enable any person skilled in the art to make and use the invention.

The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

I claim:

1. A gas delivery unit for delivering a respiratory gas of a subject comprising:
an expiratory limb for delivering an expiratory gas;
an inspiratory limb for delivering an inspiratory gas;
a common limb connecting at a branching point with said expiratory limb and said inspiratory limb for delivering both said expiratory gas and said inspiratory gas; and
at least one port for a fluid dispenser.
wherein said port is configured to open into at least one of said inspiratory limb, said expiratory limb, said common limb and said branching point, said port haying a longitudinal axis along an opening direction, and which longitudinal axis of said port is configured to form an angle δ, which is less than 90° degrees, with a longitudinal axis of one of said inspiratory limb, said expiratory limb and said common limb, and Which longitudinal axis of said inspiratory limb is configured to form an angle β, which is at an angle of 100°-180° degrees, with the longitudinal axis of said common limb.

2. The gas delivery unit of claim 1, wherein said longitudinal axis of said port along said opening direction is towards one of said inspiratory limb, said common limb and said branching point to dispense at least major part of fluid from said fluid dispenser into inspiratory gas during an inspiratory phase.

3. The gas delivery unit of claim 1, wherein said longitudinal axis of said inspiratory limb is configured to form an angle α, which is at an angle of 0°-170° degrees, with said longitudinal axis of said expiratory limb.

4. The gas delivery unit of claim 1, wherein said angle β is between 110°-160° degrees.

5. The gas delivery unit of claim 3, wherein said angle α is between 30°-120° degrees.

6. The gas delivery unit of claim 1, wherein said angle β is between 125°-145° degrees.

7. The gas delivery unit of claim 3, wherein said angle α is between 45°-100° degrees.

8. The gas delivery unit of claim 1, wherein said angle δ is between 10°-90° degrees.

9. The gas delivery unit of claim 1, wherein said longitudinal axis of said at least one port is configured to form angle γ, which is at an angle of 90° -170° degrees, with said longitudinal axis of said common limb.

10. The gas delivery unit of claim 1, wherein said angle δ is between 20°-80° degrees.

11. The gas delivery unit of claim 10, wherein said angle γ is between 100°-160° degrees.

12. The gas delivery unit of claim 11, wherein said angle δ is between 30°-70° degrees and said angle γ is between 110°-150° degrees.

13. The gas delivery unit of claim 1, wherein said longitudinal axis of said inspiratory limb and said longitudinal axis of said expiratory limb are configured to firm a first plane and said longitudinal axis of said common limb is configured to form a second plane, which second plane is at an angle τ with said first plane, said angle τ deviating from 180° degrees.

14. The gas delivery unit of claim 13, wherein said angle τ is between 90°-170° degrees, more specifically between 100°-160° degrees, or even more specifically between 120°-145° degrees.

15. The gas delivery unit of claim 1, wherein said port is configured to open through an additional limb having same longitudinal axis as said port into at least one of said inspiratory limb, said expiratory limb, said common limb and said branching point.

16. The gas delivery unit of claim 15, wherein one of said at least one port and said additional limb is configured to locate within a distance from said branching point, which is less than a diameter of said expiratory limb.

17. A breathing mask for delivering a respiratory gas of a subject comprising:
an expiratory limb for delivering an expiratory gas during an expiratory phase;
an inspiratory limb for delivering an inspiratory gas during an inspiratory phase;
a common limb connecting at a branching point with said expiratory limb and said inspiratory limb for delivering both said expiratory gas and said inspiratory gas; and
at least one port for a fluid dispenser,
wherein said port is configured to open into at least one of said inspiratory limb, said expiratory limb, said common limb and said branching point, said port having a longitudinal axis along an opening direction, and which longitudinal axis of said port is configured to form an angle δ, which is less than 90° degrees, with a longitudinal axis of one of said inspiratory limb, said expiratory limb and said common limb, and which longitudinal axis of said inspiratory limb is configured to form an angle β is at an angle of 100°-180° degrees, with the longitudinal axis of said common limb.

18. The breathing mask of claim 17. further comprising at least two respiratory tubes in operational contact with said common limb for delivering inspiratory was from said common limb into nasal cavities of the subject and expiratory _{g}as from the nasal cavities of the subject to said common limb.

19. The breathing mask of claim 17, further comprising at least one respiratory tube in operational contact with said common limb for delivering inspiratory gas from said common limb into oral cavity of the subject and expiratory gas from the oral cavity of the subject to said common limb.

20. A breathing mask for delivering a respiratory gas of a subject comprising:
an expiratory limb for delivering an expiratory gas;
an inspiratory limb for delivering an inspiratory gas;
a common limb connecting, at a branching point with said expiratory limb said inspiratory limb for delivering both said expiratory gas and said inspiratory gas;
at least one of only one respiratory tube and at least two respiratory tubes, said common limb being in operational contact with one of said only one respiratory tube and said at least two respiratory tubes for delivering inspiratory gas from said common limb into at least one cavity of the subject and expiratory gas from said at least one cavity of the subject to said common. limb; and
at least one port for a fluid dispenser,
wherein said port is configured to open into at least one of said inspiratory limb, said expiratory limb, said common limb and said branching point, said port having a longitudinal axis along an opening direction, and which longitudinal axis of said port is configured to form an angle δ, which is less than 90° degrees, with a longitudinal axis of one of said inspiratory limb, said expiratory limb and said common limb, and which longitudinal axis of said inspiratory limb is configured to form an angle β, which is at an angle of 100°-180° degrees, with the longitudinal axis of said common limb, and wherein a diameter of said common limb is configured to deviate less than 10% from one of a diameter of said only one respiratory tube and combined diameters of said at least two respiratory tubes.
